(19) United States
(12) Patent Application Publication
(10) Pub. No.: US 2011/0177017 A1
(43) Pub. Date: Jul. 21, 2011

(54) Non-Aerosol Personal Care Compositions Comprising A Hydrophobically Modified Cationic Polysaccharide

(76) Inventors: Timothy Woodrow Coffindaffer, Maineville, OH (US); Benjamin Parker Heath, Cincinnati, OH (US); Kenneth Eugene Kyte, III, Oregonia, OH (US)

(21) Appl. No.: 13/005,003

(22) Filed: Jan. 12, 2011

Related U.S. Application Data
(60) Provisional application No. 61/295,442, filed on Jan. 15, 2010.

(51) Int. Cl. A61K 8/73; A61K 8/81

(52) U.S. Cl. 424/70.13

(57) ABSTRACT

A personal care composition suitable for use as a non-aerosol shave composition comprising: a cationic polysaccharide, wherein said cationic polysaccharide is hydrophobically modified with a hydrophobic substituent and a cationic substituent; a lathering surfactant; a carrier comprising water; and optional adjunct ingredients such as lubricants.

### CROSS REFERENCE TO PENDING APPLICATIONS

This application claims the benefit of U.S. Provisional Application No. 61/295,442 filed on Jan. 15, 2010.

### BACKGROUND OF THE INVENTION

Over the past century, the forms of shave aids have changed and evolved. Shaving soaps and brushes were used in the early part of the 20^{th} century, where the soap was placed into a mug and rubbed with the brush to generate lather. After World War II, aerosol technology, developed to deliver pesticides, was reapplied and the first aerosol shave foams were born. These shave foams are composed of thin soap solutions and a volatile hydrocarbon that when mixed together in situ form a voluminous, creamy lather. Another popular form used today is the post foaming shave gel, developed in the late 70's. Amine-based soaps are combined with volatile hydrocarbons to form a clear, stable emulsion when kept under pressure. Once dispensed and mechanically agitated, these gels transform into thick foams. Although aerosols are by far the most widely used form of shave aid, they are almost exclusively based on soap technology. Soaps can be harsh to the skin, especially in hard water, and limit the types of ingredients that can be included due to the high pH of the product. Aerosol shave gels also require a package with an inner compartment, into which the shave gel is filled, and an outer chamber, which is filled with a high pressure gas that causes the shave gel to be dispensed when actuated.

Non-aerosol shave aids can be formulated over a wide pH range to allow for inclusion of skin benefit agents and can be packaged into much less expensive containers. The two main types of non-aerosol shave aids sold today are either emulsions (creams/lotions) or gels, which most commonly consist of polymer thickened surfactant systems. Most non-aerosol shave aids fall within a pH range of 5-9, which allows for the use of any number of thickeners and by extension the development of clear, translucent or opaque products. The addition of various polymers into personal care compositions is known. See e.g. U.S. Patent Publ. No. 2007/0207106; U.S. Pat. Nos. 5,902,574 and 5,262,154. The addition of many of these polymeric materials has been described to provide thickening benefits. The addition of many of these polymers, however, is believed to cause the compositions, such as non-aerosol shave gels, to become cloudy or opaque, which can be aesthetically unpleasing to some consumers. As such, there is an ongoing need for new polymers which can provide the desired viscosity or rheology, which can be used at various pH levels (including at low pH, i.e. below 4) and still fewer that can produce a thick, lubricious gel with a desirable aesthetic appearance.

### SUMMARY OF THE INVENTION

One aspect of the present invention provides for a personal care composition suitable for use as a non-aerosol shave composition comprising: from about 0.1% to about 3% of a cationic polysaccharide, wherein said cationic polysaccharide is hydrophobically modified with a hydrophobic substituent and a cationic substituent; from about 5% to about 30% of a lathering surfactant; from about 50% to about 90% of a carrier comprising water; and optional adjunct ingredients such as lubricants.

### DETAILED DESCRIPTION OF THE INVENTION

### 1. Hydrophobically Modified Cationic Polysaccharide

The personal care composition of the present invention comprises a hydrophobically modified cationic polysaccharide, modified with a hydrophobic substituent and a cationic substituent. The hydrophobically modified cationic polysaccharide is used at a level of from about 0.1% to about 3%, or from about 0.25% to about 2.5%, or from about 0.5% to about 2.3%, or from about 1% to about 2% by weight. Non-limiting examples of suitable hydrophobically modified cationic polysaccharides comprise cellulose, starch and guar derivatives, particularly a derivatized hydroxyethyl cellulose ether (such as those sold under the Trade Name of SoftCAT™).

Nonlimiting examples of hydrophobically modified quaternized hydroxyethyl cellulose ethers include: those referred to in US 2007 0031362 A1 from Union Carbide, and can be referred to by those skilled in the art as SoftCAT.

“Hydrophobically modified quaternized cellulose ethers” refers to cellulose ether derivatives containing quaternary ammonium groups. In one embodiment, the quaternized cellulose ether comprises a cellulose ether which has from 4,000 to 10,000 anhydroglucose repeat units and which is substituted with

(a) on the average from 0.0003 to 0.08 moles, per mole of anhydroglucose unit, of a substituent comprising an alkyl or arylalkyl group having from 8 to 24 carbon atoms and

(b) a substituent having the formula II

[R⁵R⁶R⁷R⁸N⁺](A^{z-})_{1z} (II)

wherein

R⁵, R⁶ and R⁷ each independently are —CH₃ or —C₂H₅,

R⁸ is —CH₂—CHOH—CH₂— or —CH₂CH₂—

A^{z-} is an anion, and

z is 1, 2 or 3.

Preferably, these cellulose ethers have an M.S. (hydroxyethyl) of from 1.0 to 3.0, more preferably from 1.5 to 2.5. The M.S. (hydroxyethyl) designates the average number of moles of hydroxyethyl groups which have been attached by an ether linkage per mole of anhydroglucose unit. The cellulose ethers have at least 4,000 anhydroglucose repeat units, preferably at least 4,500 anhydroglucose repeat units, more preferably at least 5,000 anhydroglucose repeat units, and most preferably at least 6,000 anhydroglucose repeat units. The cellulose ethers have up to 10,000 anhydroglucose repeat units, preferably up to 9,000 anhydroglucose repeat units and most preferably up to 8,000 anhydroglucose repeat units. Such cellulose ethers are readily commercially available. Alternatively, such cellulose ethers can be prepared from cellulose by methods known in the art.

Typical cellulose ethers include for example, hydroxyethyl cellulose, hydroxypropyl cellulose, methyl cellulose, hydroxypropyl methyl cellulose, hydroxyethyl methyl cellulose, hydroxyethyl carboxylmethyl cellulose, or mixtures thereof. Preferred cellulose ethers include hydroxyethyl cellulose and hydroxypropyl cellulose. Other suitable cellulose ethers comprise hydroxyethyl groups. The above cellulose ethers can be derivatized with a hydrophobic substituent and a cationic nitrogen-containing substituent to form quaternized cellulose ethers of the present invention.

The cellulose ether derivatives of the present invention are cellulose ethers which are substituted with a hydrophobic substituent (a) and a cationic substituent (b) as described below.

Hydrophobic substituents (a) suitable for use in accordance with the present invention comprise an alkyl or arylalkyl group having from 8 to 24 carbon atoms, preferably from 10 to 24 carbon atoms, more preferably from 12 to 18 carbon atoms, and most preferably 12 to 15 carbon atoms. As used herein the term “arylalkyl group” means a group containing both aromatic and aliphatic structures. The most preferred aliphatic hydrophobic substituent is the dodecyl group, which is most preferably straight-chained. The hydrophobic substituent is typically cationic or non-ionic. Many hydrophobe-containing reagents suitable for use as hydrophobic substituents are commercially available. In addition, methods for preparing such hydrophobe-containing reagents, as well as methods for derivatizing cellulose ethers to comprise such hydrophobic substituents, are known to those skilled in the art. Note for example, U.S. Pat. Nos. 4,228,277, 4,663,159 and 4,845,175.

A preferred hydrophobic substituent (a) suitable for use in accordance with the present invention has the formula (I)

R¹R²R³R⁴N⁺(A^{z-})_{1/z} (I)

wherein

R¹ and R² each independently are —CH₃ or —C₂H₅,

R³ is —CH₂—CHOH—CH₂— or —CH₂CH₂—

R⁴ is an alkyl or arylalkyl group having from 8 to 24 carbon atoms, and

A^{z-} is an anion and

z is 1, 2 or 3.

Preferably, R¹ and more preferably, both R¹ and R² are —CH₃. Preferably, R³ is —CH₂—CHOH—CH₂—. Preferably, R⁴ is —CₙH₍₂ₙ₊₁₎, where n is from 8 to 24, more preferably from 10 to 18, most preferably 12. A^{z-} is an anion with the valency of z, such as phosphate, nitrate, sulfate or halide. Chloride is the most preferred ion. Z is preferably 1 or 2, more preferably 1. The most preferred hydrophobic substituents (a) are those wherein two or more, preferably each of R′, R², R³, R⁴, A^{z-} and z have the mentioned preferred meanings.

Other preferred hydrophobic substituents include those derived from hydrophobe-containing reagents comprising alkyl or arylalkyl groups having from 8 to 24 carbon atoms, preferably from 10 to 24 carbon atoms, more preferably from 12 to 18 carbon atoms, and most preferably 12 to 15 carbon atoms. Preferred are glycidyl ethers, such, as nonylphenyl glycidyl ether or dodecylphlelnl glycidyl ether; or alpha-olefin epoxides, such as 1,2-epoxy hexadecane and their respective chlorohydrins, or alkyl halides, e.g., dodecyl bromide, and mixtures thereof.

The average substitution level of the substituent (a) is at least 0.0003, preferably at least 0.0005 moles per mole of anhydroglucose unit and up to 0.08, preferably up to 0.07, and most preferably up to 0.05 moles per mole of anhydroglucose unit. More than one particular hydrophobic substituent can be substituted onto the cellulose ether provided that the total substitution level is within the ranges set forth above.

The cationic substituent (b) suitable for use in accordance with the present invention has the formula II (above). Preferably, R⁵ is —CH₃. More preferably, R⁵, R⁶ and R⁷ are —CH₃. Preferably, R⁸ is —CH₂—CHOH—CH₂—. A^{z-} is an anion with the valency of z, such as phosphate, nitrate, sulfate or halide. Chloride is the most preferred ion. Z is preferably 1 or 2, more preferably 1. The most preferred cationic substituents (b) are those wherein two or more, preferably each of R⁵, R⁶, R⁷, R⁸, A^{z-} and z have the mentioned preferred meanings.

Preferred quaternized cellulose polymers with hydrophobic substitution are referred to in the industry Personal Care Products Council (formerly the Cosmetic, Toiletry, and Fragrance Association) as Polyquaternium-67 and are available from Dow Chemical (Amerchol Corp.) under the tradename SoftCAT™ which includes their SL, SX, and SK series polymers.

### 2. Lathering Surfactants

The personal care composition, which can be a wash or cleansing composition (such as a shave prep composition), can comprise one or more lathering surfactants and a carrier such at water, at a total level of from about 60% to about 99.99%. A lathering surfactant defined herein as surfactant, which when combined with water and mechanically agitated generates a foam or lather. Preferably, these surfactants or combinations of surfactants should be mild, which means that these surfactants provide sufficient cleansing or detersive benefits but do not overly dry the skin or hair while still being able to produce a lather.

A wide variety of lathering surfactants are useful herein and include those selected from the group consisting of anionic lathering surfactants, nonionic lather surfactants, amphoteric lathering surfactants, and mixtures thereof. Generally, the lathering surfactants are fairly water soluble. When used in the composition, at least about 4% of the lathering surfactants have a HLB value greater than about ten. Examples of such surfactants are found in and U.S. Pat. No. 5,624,666. Cationic surfactants can also be used as optional components, provided they do not negatively impact the overall lathering characteristics of the required lathering surfactants.

Concentrations of these surfactant are from about 10% to about 20%, alternatively from about 5% to about 25%, and alternatively from about 2% to about 30% by weight of the composition. To avoid skin irritation issues, the compositions containing anionic surfactants should have a ratio by weight of the composition of anionic surfactant to amphoteric and/or zwitterionic surfactant is from about 1.1:1 to about 1:1.5, alternatively from about 1.25:1 to about 1:2, and alternatively from about 1.5:1 to about 1:3.

Anionic lathering surfactants useful in the compositions of the present invention are disclosed in McCutcheon's, Detergents and Emulsifiers, North American edition (1986), published by allured Publishing Corporation; McCutcheon's, Functional Materials, North American Edition (1992); and U.S. Pat. No. 3,929,678. A wide variety of anionic lathering surfactants are useful herein. Non-limiting examples of anionic lathering surfactants include those selected from the group consisting of sarcosinates, sulfates, sulfonates, isethionates, taurates, phosphates, lactylates, glutamates, and mixtures thereof.

Other anionic materials useful herein are soaps (i.e., alkali metal salts, e.g., sodium or potassium salts) of fatty acids, typically having from about 8 to about 24 carbon atoms, preferably from about 10 to about 20 carbon atoms, monoalkyl, dialkyl, and trialkylphosphate salts, alkanoyl sarcosinates corresponding to the formula RCON(CH₃)CH₂CH₂CO₂M wherein R is alkyl or alkenyl of about 10 to about 20 carbon atoms, and M is a water-soluble cation such as ammonium, sodium, potassium and alkanolamine (e.g., triethanolamine). Also useful are to taurates which are based on taurine, which is also known as 2-aminoethanesulfonic acid, and glutamates, especially those having carbon chains between C₈ and C₁₆.

Non-limiting examples of preferred anionic lathering surfactants useful herein include those selected from the group consisting of sodium lauryl sulfate, ammonium lauryl sulfate, ammonium laureth sulfate, sodium laureth sulfate, sodium trideceth sulfate, ammonium cetyl sulfate, sodium cetyl sulfate, ammonium cocoyl isethionate, sodium lauroyl isethionate, sodium lauroyl lactylate, triethanolamine lauroyl lactylate, sodium caproyl lactylate, sodium lauroyl sarcosinate, sodium myristoyl sarcosinate, sodium cocoyl sarcosinate, sodium lauroyl methyl taurate, sodium cocoyl methyl taurate, sodium lauroyl glutamate, sodium myristoyl glutamate, and sodium cocoyl glutamate and mixtures thereof.

Suitable amphoteric or zwitterionic detersive surfactants for use in the compositions herein include those which are known for use in hair care or other personal care cleansing. Concentration of such amphoteric detersive surfactants can be from about 1% to about 10%, alternatively from about 0.5% to about 20% by weight of the composition. Non-limiting examples of suitable zwitterionic or amphoteric surfactants are described in U.S. Pat. Nos. 5,104,646 and 5,106,609.

Nonionic lathering surfactants for use in the compositions of the present invention are disclosed in McCutcheon's, Detergents and Emulsifiers, North American edition (1986), published by allured Publishing Corporation; and McCutcheon's, Functional Materials, North American Edition (1992); both of which are incorporated by reference herein in their entirety. Nonionic lathering surfactants useful herein include those selected from the group consisting of alkyl glucosides, alkyl polyglucosides, polyhydroxy fatty acid amides, alkoxylated fatty acid esters, lathering sucrose esters, amine oxides, and mixtures thereof.

Other examples of nonionic surfactants include amine oxides. Amine oxides correspond to the general formula R¹R²R³NO, wherein R¹ contains an alkyl, alkenyl or monohydroxy alkyl radical of from about 8 to about 18 carbon atoms, from 0 to about 10 ethylene oxide moieties, and from 0 to about 1 glyceryl moiety, and R² and R³ contain from about 1 to about 3 carbon atoms and from 0 to about 1 hydroxy group, e.g., methyl, ethyl, propyl, hydroxyethyl, or hydroxypropyl radicals. Examples of amine oxides suitable for use in this invention include dimethyl-dodecylamine oxide, oleyldi(2-hydroxyethyl) amine oxide, dimethyloctylamine oxide, dimethyl-decylamine oxide, dimethyl-tetradecylamine oxide, 3,6,9-trioxaheptadecyldiethylamine oxide, di(2-hydroxyethyl)-tetradecylamine oxide, 2-dodecoxyethyldimethylamine oxide, 3-dodecoxy-2-hydroxypropyldi(3-hydroxypropyl)amine oxide, dimethylhexadecylamine oxide.

Preferred lathering surfactants for use herein are the following, wherein the anionic lathering surfactant is selected from the group consisting of ammonium lauroyl sarcosinate, sodium trideceth sulfate, sodium lauroyl sarcosinate, sodium myristoyl sarcosinate, ammonium laureth sulfate, sodium laureth sulfate, ammonium lauryl sulfate, sodium lauryl sulfate, ammonium cocoyl isethionate, sodium cocoyl isethionate, sodium lauroyl isethionate, sodium cetyl sulfate, sodium lauroyl lactylate, triethanolamine lauroyl lactylate, and mixtures thereof; wherein the nonionic lathering surfactant is selected from the group consisting of lauramine oxide, cocoamine oxide, decyl polyglucose, lauryl polyglucose, sucrose cocoate, C₁₂₋₁₄ glucosamides, sucrose laurate, and mixtures thereof; and wherein the amphoteric lathering surfactant is selected from the group consisting of disodium lauroamphodiacetate, sodium lauroamphoacetate, cetyl dimethyl betaine, cocoamidopropyl betaine, coco-betaine, cetyl betaine, lauramidopropyl betaine, cocoamidopropyl hydroxy sultaine, and mixtures thereof.

One suitable lathering surfactant is a polyglyceryl fatty ester. In one embodiment the polyglyceryl fatty ester surfactant has the formula:

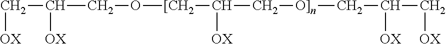

wherein n is 1 to 10, and X is a hydrogen atom or a long chain acyl group derived from a C₁₂₋₂₂ fatty acid or an N-fatty acyl-neutral amino acid, provided that at least one X is a long chain acyl group and no more than three X's are long chain acyl groups. In one embodiment, the polyglyceryl fatty ester surfactant is selected from the group consisting of: polyglyceryl-10 oleate, polyglyceryl-6 stearate, polyglyceryl-10 stearate, polyglyceryl-8 dipalmitate, polyglyceryl-10 dipalmitate, polyglyceryl-10 behenate, and polyglyceryl-12 trilaurate.

### 3. Carrier

The personal care compositions of the present invention also comprise a carrier. In one embodiment the carrier comprises water. The carrier is preferably dermatologically acceptable, meaning that the carrier is suitable for topical application to the keratinous tissue, has good aesthetic properties, is compatible with the actives of the present invention and any other components, and will not cause any safety or toxicity concerns. In one embodiment, the personal care composition comprises from about 50% to about 99.99%, preferably from about 60% to about 99.9%, more preferably from about 70% to about 98%, and even more preferably from about 80% to about 95% of the carrier by weight of the composition.

### 4. Adjunct Ingredients

a. Lubricants

In one embodiment, said personal care composition comprises at least one lubricant selected from: a lubricious water soluble polymer; a water insoluble particle, a hydrogel forming polymer, and a mixture thereof.

The lubricious water soluble polymer will generally have a molecular weight greater between about 300,000 and 15,000,000 daltons, preferably more than about one million daltons, and will include a sufficient number of hydrophilic moieties or substituents on the polymer chain to render the polymer water soluble. The polymer may be a homopolymer, copolymer or terpolymer. Examples of suitable lubricious water soluble polymers include polyethylene oxide, polyvinylpyrrolidone, and polyacrylamide. A preferred lubricious water soluble polymer comprises polyethylene oxide, and more particularly a polyethylene oxide with a molecular weight of about 0.5 to about 5 million daltons. Examples of suitable polyethylene oxides PEG-23M, PEG-45M, and PEG-90M. The lubricious water soluble polymer can be at a level of about 0.005% to about 3%, preferably about 0.01% to about 1%, by weight.

The water insoluble particles may include inorganic particles or organic polymer particles. Examples of inorganic particles include titanium dioxide, silicas, silicates and glass beads, with glass beads being preferred. Examples of organic polymer particles include polytetrafluoroethylene particles, polyethylene particles, polypropylene particles, polyurethane particles, polyamide particles, or mixtures of two or more of such particles.

The hydrogel-forming polymer is a highly hydrophilic polymer that, in water, forms organized three-dimensional domains of approximately nanometer scale. The hydrogel-forming polymer generally has a molecular weight greater than about one million daltons (although lower molecular weights are possible) and typically is at least partially or lightly crosslinked and may be at least partially water insoluble, but it also includes a sufficient number of hydrophilic moieties so as to enable the polymer to trap or bind a substantial amount of water within the polymer matrix and thereby form three-dimensional domains. Generally, the hydrogel-forming polymer will be included in the shaving composition in an amount of about 0.0005% to about 3%, or about 0.001% to about 0.5%, or about 0.002% to about 0.1%, by weight.

Examples of suitable hydrogel-forming polymers include a polyacrylic acid or polymethacrylic acid partially esterified with a polyhydric alcohol; hydrophilic polyurethanes; lightly crosslinked polyethylene oxide; lightly crosslinked polyvinyl alcohol; lightly crosslinked polyacrylamide; hydrophobically modified hydroxyalkyl cellulose; hydroxyethyl methacrylate; and crosslinked hyaluronic acid. A preferred hydrogel-forming polymer comprises polyacrylic acid partially esterified (e.g., about 40% to 60%, preferably about 50%, esterified) with glycerin. Such a polymer includes glyceryl acrylate/acrylic acid copolymer. Glyceryl acrylate/acrylic acid copolymer is highly hydrophilic, has a molecular weight greater than 1 million daltons and generally includes a polyacrylic acid backbone partially esterified (typically about 50% esterified) with glycerin. It is believed that the glyceryl acrylate/acrylic acid copolymer forms a clathrate that holds water, which, upon release, supplies lubrication and moisturization to the skin It has been found that shave gel compositions that include the glyceryl acrylate/acrylic acid copolymer have improved gel structure and reduced coefficient of friction (i.e., increased lubricity). See e.g. U.S. 2006/00257349 at ¶ 110.

The term “water dispersible”, as used herein, means that a substance is either substantially dispersible or soluble in water. The water dispersible surface active agent is preferably one that is capable of forming a lather, such as one or more of the optional lathering surfactants described in section 5 below (including but not limited to a soap, an interrupted soap, a detergent, an anionic surfactant, a non-ionic surfactant or a mixture of one or more of these.)

b. Polar Solvents

In one embodiment, the carrier comprises a polar solvent. The level of polar solvent can be from about 1% to about 20%, or from about 5% to about 10%. Polar solvents useful herein include polyhydric alcohols such as, 3-butylene glycol, propane diol, ethylene glycol, diethylene glycol, sorbitol, and other sugars which are in liquid form at ambient temperature glycerin, sorbitol, propylene glycol, butylene glycol, pentylene glycol, hexylene glycol, ethoxylated glucose, 1,2-hexane diol, hexanetriol, dipropylene glycol, erythritol, trehalose, diglycerin, xylitol, maltitol, maltose, glucose, fructose, sodium chondroitin sulfate, sodium hyaluronate, sodium adenosine phosphate, sodium lactate, pyrrolidone carbonate, glucosamine, cyclodextrin, and mixtures thereof. Polyols such as those containing from 2 to about 6 carbon atoms and from 2 to about 6 hydroxy groups are preferred (e.g., 1,3-propanediol, ethylene glycol, glycerin, and 1,2-propanediol) can also be used. The most preferred are Butylene, Pentylene or Hexylene Glycol and mixtures thereof.

Without intending to be bound by theory, it is believed that the addition of one or more, polar solvents, allows for reduction in the viscosity and improvement in the clarity of the personal care composition while maintaining good lubrication.

c. Salycylic Acid

The personal care composition of the present invention may comprise a salicylic acid compound, its esters, its salts, or combinations thereof. In the compositions of the present invention, the salicylic acid compound preferably comprises from about 0.1% to about 5%, preferably from about 0.2% to about 2%, and more preferably from about 0.5% to about 2%, by weight of the composition, of salicylic acid.

d. Other Adjunct Ingredients

The compositions of the present invention may contain a variety of other ingredients that are conventionally used in given product types provided that they do not unacceptably alter the benefits of the invention. These ingredients should be included in a safe and effective amount for a personal care composition for application to skin.

The CTFA Cosmetic Ingredient Handbook, Second Edition (1992) describes a wide variety of nonlimiting cosmetic and pharmaceutical ingredients commonly used in the skin care industry, which are suitable for use in the compositions of the present invention. Examples of these ingredient classes include: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, fatty alcohols and fatty acids, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents, skin-conditioning agents, skin soothing and/or healing agents and derivatives, skin treating agents, thickeners, and vitamins and derivatives thereof.

Additional non-limiting examples of additional suitable skin treatment actives are included in U.S. 2003/0082219 in Section I (i.e. hexamidine, zinc oxide, and niacinamide); U.S. Pat. No. 5,665,339 at Section D (i.e. coolants, skin conditioning agents, sunscreens and pigments, and to medicaments); and US 2005/0019356 (i.e. desquamation actives, anti-acne actives, chelators, flavonoids, and antimicrobial and antifungal actives). Other useful optional ingredients include: Anti-Wrinkle Actives and/or Anti-Atrophy Actives; Anti-Oxidants and/or Racial Scavengers; Anti-Inflammatory Agents; Anti-Cellulite Agents; Tanning Actives; Skin Lightening Agents; Sunscreen Actives; Water Soluble Vitamins; particulates; and combinations thereof. The personal care composition of the present invention is a non-aerosol composition. In one embodiment, the personal care composition is free or substantially free of a volatile post-foaming agent. Substantially free means that no amount of the ingredient is intentionally added to the composition but trace amounts may be present as carryover from processing.

i. Conditioning Agents

The compositions of the present invention may comprise a conditioning agent selected from the group consisting of humectants, moisturizers, or skin conditioners, each can be present at a level of from about 0.01% to about 40%, more preferably from about 0.1% to about 30%, and even more preferably from about 0.5% to about 15% by weight of the composition. These materials include, but are not limited to, guanidine; urea; glycolic acid and glycolate salts (e.g. ammonium and quaternary alkyl ammonium); lactic acid and lactate salts (e.g., ammonium and quaternary alkyl ammonium); aloe vera in any of its variety of forms (e.g., aloe vera gel); polyhydroxy compounds such as sorbitol, mannitol, glycerol, hexanetriol, butanetriol, propylene glycol, butylene glycol, hexylene glycol and the like; polyethylene glycols; sugars (e.g., melibiose) and starches; sugar and starch derivatives (e.g., alkoxylated glucose, fructose, sucrose, etc.); hyaluronic acid; lactamide monoethanolamine; acetamide monoethanolamine; sucrose polyester; petrolatum; and mixtures thereof.

Suitable moisturizers, also referred to in the present invention as humectants, include urea, guanidine, glycolic acid and glycolate salts (e.g. ammonium and quaternary alkyl ammonium), lactic acid and lactate salts (e.g. ammonium and quaternary alkyl ammonium), aloe vera in any of its variety of forms (e.g. aloe vera gel), polyhydroxy alcohols (such as sorbitol, glycerol, hexanetriol, propylene glycol, hexylene glycol and the like), polyethylene glycol, sugars and starches, sugar and starch derivatives (e.g. alkoxylated glucose), hyaluronic acid, lactamide monoethanolamine, acetamide monoethanolamine, and mixtures thereof.

iI. Thickening Agents (Including Thickeners and Gelling Agents)

The compositions of the present invention can comprise one or more thickening agents, to preferably from about 0.05% to about 10%, more preferably from about 0.1% to about 5%, and even more preferably from about 0.25% to about 4%, by weight of the composition. Nonlimiting classes of thickening agents include those selected from the group consisting of: Carboxylic Acid Polymers (crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol); crosslinked polyacrylate polymers (including both cationic and nonionic polymers, such as described in U.S. Pat. Nos. 5,100,660; 4,849,484; 4,835,206; 4,628,078; 4,599,379, and EP 228,868); polymeric sulfonic acid (such as copolymers of acryloyldimethyltaurate and vinylpyrrolidone) and hydrophobically modified polymeric sulfonic acid (such as crosspolymers of acryloyldimethyltaurate and beheneth-25 methacrylate); polyacrylamide polymers (such as nonionic polyacrylamide polymers including substituted branched or unbranched polymers such as polyacrylamide and isoparaffin and laureth-7 and multi-block copolymers of acrylamides and substituted acrylamides with acrylic acids and substituted acrylic acids); polysaccharides (nonlimiting examples of polysaccharide gelling agents include those selected from the group consisting of cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof); gums (i.e. gum agents such as acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof); and crystalline, hydroxyl-containing fatty acids, fatty esters or fatty waxes (such as microfibrous bacterial cellulose structurants as disclosed in U.S. Pat. Nos. 6,967,027 to Heux et al.; 5,207,826 to Westland et al.; 4,487,634 to Turbak et al.; 4,373,702 to Turbak et al. and 4,863,565 to Johnson et al., U.S. Patent Publ. No. 2007/0027108 to Yang et al.)

### 5. Compositional pH

The personal care composition of the present invention preferably has a pH of less than about 9, more preferably less than about 7. In one embodiment, the composition has a pH of less than about 10, more preferably less than about 9.5 and most preferably less than about 9. In one embodiment the composition has a pH of less than about 5, or less than about 4. In one preferred embodiment the composition has a pH range of from about 2.5 to about 4.5 Suitable lathering surfactants for use at pH levels below about 4 can be selected from the group consisting of alkyl sulfonates, pareth sulfonates, sulfobetaines, alkylhydroxysultaines, alkylglucosides and mixtures thereof.

### 6. Methods of Use

The present composition is preferably a hair removal preparation such as a non-aerosol shave foam or gel. The personal care composition of the present invention can be used in combination with various hair removal applications (prior to, concurrently with, and/or after), including but not limited to shaving (wet or dry shaving, via electric razors, via powered or manual razors which can be reuseable or disposable, and combinations thereof), epilation, electrolysis, wax or depilatories as well as energy delivery devices to help regulate hair growth. Nonlimiting examples of energy deliver devices include: light, heat, sound (including ultrasonic waves and radio frequency), electrical energy, magnetic energy, electromagnetic energy (including radiofrequency waves and microwaves), and combinations thereof. The light energy may be delivered by devices including, but not limited to, lasers, diode lasers, diode laser bars, diode laser arrays, flash lamps, intense pulsed light (IPL) sources, and combinations thereof. See e.g. US2006/0235370A1.

The present invention includes a method of treating skin irritation which can be the result of one or more of said hair removal technologies, said method comprising: applying a personal care composition to a portion of skin to form a portion of treated skin, said personal care composition comprising a naphthalenyl ketone; and down regulating at least one pain receptor in proximity with said portion of treated skin. In one embodiment, said method further comprises a step of at least partially removing hair from said portion of skin prior to the step of applying the personal care composition. In another embodiment, said method further comprises a step of at least partially removing hair from said portion of treated skin after to the step of applying the personal care composition.

### 7. Methods of Making

One embodiment of the present invention provides for a method of making the personal care composition. One method for making the present composition comprises:
a. Weighing out the water in a vessel sufficient to hold the entire batch
b. Inserting an overhead mixer with impeller into the vessel and increase agitation to create a vortex
c. Pre-blending the cationic polysaccharide and PEG polymer powders
d. Adding the polymer blend into the vortex until incorporated (addition can be done by sprinkling)
e. Begin heating batch to 70 C to hydrate the polymers, increasing rpms to maintain good mixing
f. Once the batch is at 70 C, adding the surfactant(s) and mix until uniform
g. Begin cooling batch to below 45 C
h. Once below 45 C, adding the perfume, preservatives, glycerin/glycols and other temperature-sensitive additives
i. Cooling to below 35 C and QS with water
   For product with acne control actives, add the salicylic acid during STEP f. addition.
   For product with water-soluble actives, add to the batch during STEP h. additions
   For product with oil-soluble actives, add to the batch during STEP f. additions

### 8. Composition Characteristics

a. Turbidity

In one embodiment, the personal care composition comprises a turbidity of below about 320 NTU, alternatively less than about 250 NTU, alternatively less than about 200 NTU, alternatively less than about 150 NTU, alternatively less than about 100 NTU, as measured by Turbimeter test method disclosed herein. Compositions with a turbidity below about 150, alternatively below about 100 are considered “clear” while those with a turbidity below about 320, alternatively below about 250 are “translucent.”

As used herein, turbidity is determined using a Hach Model 2100AN Turbidimeter (“Turbimeter”), by Hach Company, Loveland, Colo. StablCal is a trademark of Hach Company.

Turbidimeter Turbidity Method: The Turbidimeter measures the turbidity from 0.1 NTU to 7500 NTU. The Turbidimeter operates on the nephelometric principle of turbidity measurement. The Turbidimeter's optical system includes a tungsten-filament lamp, a 90° detector to monitor scattered light and a transmitted light detector. The Turbidimeter's microprocessor calculates the ratio of the signals from the 90° and of transmitted light detectors. This ratio technique corrects for the interferences from color and or light absorbing materials and compensates for fluctuations in the lamp intensity.

Calibration is by StablCal® Secondary standards provided with the Turbidimeter. The undiluted sample is contained in the sample cell, the outer cell wall is wiped free of water and finger prints. A thin coat of silicone oil is applied to the outer wall of the sample cell in order to mask minor imperfections and scratches on the sample cell wall, which may contribute to turbidity or stray light. A measurement is taken and result is displayed in NTU units. All samples are equilibrated and measured at 25° C. The samples are measured within 24 h after making

b. Viscosity

Viscosity measurements are determined using a Brookfield viscometer with Helipath using a spindle T-C @ 5 rpms. Sample product is poured into a glass jar having dimensions of about 11 cm high by 5 cm wide and is filled to a depth of at least 8 cm and allowed to set for 24 hours prior to measurement. The T-C spindle was attached to the viscometer and placed over the top of the sample. The Helipath is activated and the spindle was slowly lowered into the product while rotating at 5 rpms. The viscosity is monitored as the spindle continued further into the product to check for uniformity of the sample. The final viscosity reading is taken as the spindle was approximately halfway through the sample.

c. In Shave Lubrication Test

It has been found that the personal care composition of the present invention provides for an in shave lubrication benefit as shown by reduced friction as measured by the In Shave Lubrication “ISL” Test defined herein. Reducing friction is important during the shave because a high friction skin surface results in bulging of the skin. When the skin bulges, the blade is more likely to engage the skin, increasing the chance for skin irritation. Therefore, by reducing friction the product helps protect the skin. In addition, lower friction results in less drag on the skin, which can also be a potential source of irritation. This method enables measurement of the coefficient of friction (CoF) of a shave preparation.

In Shave Lubrication Test Method: An apparatus designed to simulate lubrication during the shaving process is connected to an instrument capable of measuring frictional forces (for example, an Instron-type instrument) and containing a load cell of about 1 kg to about 100 kg. The rinsing apparatus comprises: 1) an air-activated clamping device capable of opening and closing to deliver pressures of about 10 psi to about 70 psi to simulate the pressure exerted by hands on hair during rinsing 2) keratinous tissue models as described herein affixed to two opposing sides of the clamping device and 3) one or more spray nozzles capable of delivering water flow rates of from about 50 ml/min. to about 1000 mL/min, for simulating shower conditions.

Procedure: Attach the rinsing apparatus to the base of a Stable Micro Systems TA XT Plus™ Texture Analyzer (TA) equipped with a 30 kg load cell, centering or aligning the clamps perpendicular to the load cell. Adjust water flow rate to approximately 200 ml/min and the water temperature to 103° F. +/−2° F. Set the air pressure for the TA clamps to approximately 30 psi. Set the instrument measurement settings as follows: TA settings, tension compression, test speed-10.0 mm/sec for 130 mm long pull. Set the macro for a total of 10 strokes. Run the first five strokes without the water on, then manually turn on the water for 2 min 15 sec for the second five strokes. During the test, data (g of force) will only be collected during the upward pull of the treated KTM, not on the return. Cover the pads on both the front and back side of the piston with a polyurethane skin pad (see JP2006233367 for details).

Wet a 2 inch by 9 inch piece of nonwoven KTM under hot (˜103° F. +/−2° F.) tap water for 30 seconds. Place 2 g+/−0.1 g of aerosol shave gel or 1 g +/−0.1 g of aerosol shave foam onto the nonwoven KTM and gently lather and spread by hand for 30 seconds. Rub excess foam on the back of the nonwoven KTM. Load the nonwoven KTM into the TA and start the test macro. At the end of the fifth stroke, turn on the rinse water. Initiate a test sequence which 1) instructs the instrument to raise the load cell to which the KTM is attached, at a rate of about 10 mm/sec 2) opens the clamps, and 3) instructs the instrument to lower the load cell. Repeat this sequence until a predetermined number of sequences may be executed, for example, 10. Between each sample, wipe the polyurethane skin pads with a piece of nonwoven KTM and an alcohol wipe to remove any possible build-up from the previous test. By calculating the total friction in grams of force (or other suitable unit of force) for dry friction and rinse friction, products may be ranked relative to each other to assess which products would be expected to have the most pleasant feel.

“KTM” as defined herein means a “Keratinous tissue mimic” which refers to one or more artificial substrates which may have one or more physical properties representative of keratinous tissue. The KTM used for the purposes of this application is TENCEL from Lenzing, Inc. Additional details on other KTMs is disclosed in Section I of U.S. Ser. No. 61/239,908 to Battaglia et al, filed Sep. 4, 2009.

### 9. Examples

The following samples are made according to the above Method of Making disclosure. QS means quantity sufficient to reach 100%. All values are percent by weight.

Table A: Comparative Examples. Samples with varying types of cationic polymer. Sample 6 is within the scope of the present invention and demonstrates desirable compositional characteristics observed when Polyquaternium-67, a hydrophobically modified cationic polysaccharide with a hydrophobic substituent and a cationic substituent is included into a personal care composition. It has importantly been found that in these samples, the desired compositional characteristics were not observed with other cationic polymers (see Samples 1-5). Viscosity, In Shave Lubrication, and turbidity measurements are made in accordance with the test methods described in Section 8, above. Applicants have found that compositions comprising polyquaternium 67 provides sufficient thickness as shown by a high viscosity, sufficient in shower lubrication as shown by a low ISL value, and composition clarity with a low turbidity value relative to other cationic polymers.

| Ingredient | Sample 1 | Sample 2 | Sample 3 | Sample 4 | Sample 5 | Sample 6 |
|---|---|---|---|---|---|---|
| Water | QS | QS | QS | QS | QS | QS |
| Salicylic Acid | 2.000% | 2.000% | 2.000% | 2.000% | 2.000% | 2.000% |
| Decyl Glucoside | 12.000% | 12.000% | 12.000% | 12.000% | 12.000% | 12.000% |
| Polyquaternium- | 2.000% | — | — | — | — | — |
| 10 (Polymer LR- | | | | | | |
| 30M) | | | | | | |
| Polyquaternium- | — | — | — | — | — | 2.000% |
| 67 (SoftCAT SK- | | | | | | |
| MH) | | | | | | |
| Cationic Guar (N- | — | 2.000% | — | — | — | — |
| Hance 3270) | | | | | | |
| Cationic Guar | — | — | 2.000% | — | — | — |
| (Aqualon AQU | | | | | | |
| D-4051) | | | | | | |
| Cationic Guar (N- | — | — | — | 2.000% | — | — |
| Hance 3196) | | | | | | |
| Cationic Guar (N- | — | — | — | — | 2.000% | — |
| Hance 3215) | | | | | | |
| Viscosity (cps) | 134,100 | 1406 | 103,800 | 287,000 | 423,800 | 331,300 |
| ISL - Final | 728 | 1157 | 771 | 565 | 658 | 513 |
| Force (g) | | | | | | |
| Turbidity (NTU) | 85.1 | 183.0 | 234.0 | 317.0 | 244.0 | 15.9 |

Samples 7-33 as shown in the below Tables B thru H provide non-limiting examples of personal care compositions in accordance with the present invention.

Table B: Samples with varying types of polar solvents. Samples 7-9 are made according to the below formulations. Viscosity measurements are made in accordance with the above viscosity test method.

| Ingredient | Sample 7 | Sample 8 | Sample 9 |
|---|---|---|---|
| Water | QS | QS | QS |
| Salicylic Acid | 2.00% | 2.00% | 2.00% |
| Decyl Glucoside | 12.00% | 12.00% | 12.00% |
| Polyquaternium-67 | 1.50% | 1.50% | 1.50% |
| (SoftCAT SL-60) | | | |
| Citric Acid | 0.60% | 0.60% | 0.60% |
| Butylene Glycol | — | 10.00% | — |
| Pentylene Glycol | — | — | 10.00% |
| Viscosity (cps) | 243400 | 145400 | 107800 |

Table C: Additional samples with varying polar solvents. Samples 10-14 are made according to the below formulations. Viscosity measurements are made in accordance with the above viscosity test method.

| | Sample | Sample | Sample | Sample | Sample |
|---|---|---|---|---|---|
| Ingredient | 10 | 11 | 12 | 13 | 14 |
| Water | QS | QS | QS | QS | QS |
| Salicylic Acid | 2.00% | 2.00% | 2.00% | 2.00% | 2.00% |
| Decyl Glucoside | 12.00% | 12.00% | 12.00% | 12.00% | 12.00% |
| Polyquaternium-67 (SoftCAT | 1.50% | 1.25% | 1.25% | 1.25% | 1.25% |
| SL-60) | | | | | |
| Polyquaternium-67 (SoftCAT | — | 0.25% | 0.25% | 0.25% | 0.25% |
| SX-400X) | | | | | |
| Citric Acid | 0.60% | 0.60% | 0.60% | 0.60% | 0.60% |
| Pentylene Glycol | 10.00% | — | 10.00% | — | — |
| Hexylene Glycol | — | — | — | — | 10.00% |
| 1,2-Hexanediol | — | — | — | 10.00% | |
| Viscosity (cps) | 107800 | 155400 | 57160 | 62270 | 68310 |

Table D: Samples with varying levels of pentylene glycol. Samples 15-19 are made according to the below formulations. Viscosity and turbidity measurements are made in accordance with test Viscosity and Turbidity test methods defined herein, respectively.

| | Sample | Sample | Sample | Sample | Sample |
|---|---|---|---|---|---|
| Ingredient | 15 | 16 | 17 | 18 | 19 |
| Water | QS | QS | QS | QS | QS |
| Salicylic Acid | 1.00% | 0.99% | 0.98% | 0.96% | 0.92% |
| Decyl | 6.00% | 5.94% | 5.88% | 5.76% | 5.52% |
| Glucoside | | | | | |
| Poly- | 1.50% | 1.49% | 1.47% | 1.44% | 1.38% |
| quaternium-67 | | | | | |
| (SoftCAT | | | | | |
| SL-5) | | | | | |
| Citric Acid | 0.60% | 0.59% | 0.59% | 0.58% | 0.55% |
| Pentylene | — | 1.000% | 2.000% | 4.000% | 8.000% |
| Glycol | | | | | |
| Fragrance | 0.50% | 0.50% | 0.49% | 0.48% | 0.46% |
| Viscosity (cps) | 84200 | 72140 | 63660 | 54550 | 47730 |
| Turbidity | 46.2 | 28.8 | 19.3 | 10.1 | 3.7 |
| (NTU) | | | | | |

Table E: Samples with varying surfactants. Samples 20-23 are made according to the below formulations.

| Ingredient | Sample 20 | Sample 21 | Sample 22 | Sample 23 |
|---|---|---|---|---|
| Water | QS | QS | QS | QS |
| Salicylic Acid | 1.00% | — | 1.00% | — |
| PEG-23M | 0.50% | 0.50% | 0.50% | 0.50% |
| Decyl Glucoside | 6.00% | 6.00% | — | — |
| Sodium C14-16 Olefin | — | — | 4.08% | 4.08% |
| Sulfonate | | | | |
| Sodium C12-15 | — | — | 1.33% | 1.33% |
| Pareth-15 Sulfonate | | | | |
| Cocamidopropyl | — | — | 3.11% | 3.11% |
| Hydroxysultaine | | | | |
| Polyquaternium-67 | 1.50% | 1.50% | 1.50% | 1.50% |
| (SoftCAT SL-5) | | | | |
| Sodium Hydroxide | — | — | 0.08% | — |
| Glycerin | 5.00% | 5.00% | 5.00% | 5.00% |
| Pentylene Glycol | 5.00% | 5.00% | 5.00% | 5.00% |
| Fragrance | 0.50% | 1.00% | 0.50% | 1.00% |

Table F: Samples with varying hydrophobically modified cationic polysaccharide. Samples 24-28 are made according to the below formulations.

| | Sample | Sample | Sample | Sample | Sample |
|---|---|---|---|---|---|
| Ingredient | 24 | 25 | 26 | 27 | 28 |
| Water | QS | QS | QS | QS | QS |
| Salicylic Acid | 1.50% | 1.50% | 1.50% | 1.50% | 1.50% |
| Sodium C14-16 Olefin | 6.18% | 6.18% | 6.18% | 6.18% | 6.18% |
| Sulfonate | | | | | |
| Sodium C12-15 | 2.01% | 2.01% | 2.01% | 2.01% | 2.01% |
| Pareth-15 Sulfonate | | | | | |
| Cocamidopropyl | 4.72% | 4.72% | 4.72% | 4.72% | 4.72% |
| Hydroxysultaine | | | | | |
| SoftCAT SK-MH | 1.00% | — | — | — | — |
| SoftCAT SL-100 | — | — | — | — | 1.00% |
| SoftCAT SL-60 | — | — | — | 1.00% | — |
| SoftCAT SL-30 | — | — | 1.00% | — | — |
| SoftCAT SL-5 | — | 1.00% | — | — | — |
| Sodium Hydroxide | — | 0.08% | 0.08% | 0.08% | 0.08% |

Table G: Samples with nonionic surfactants. Samples 29-30 are made according to the below formulations.

| Ingredient | Sample 29 | Sample 30 |
|---|---|---|
| Water | QS | QS |
| Salicylic Acid | 2.00% | 2.00% |
| Decyl Glucoside | 12.00% | 12.00% |
| Polyquaternium-67 (SoftCAT SL-60) | 1.50% | — |
| Polyquaternium-67 (SoftCAT SL-5) | — | 1.50% |
| Citric Acid | 0.60% | 0.60% |

Table H: Samples with varying levels of nonionic surfactant. Samples 31-33 are made according to the below formulations.

| Ingredient | Sample 31 | Sample 32 | Sample 33 |
|---|---|---|---|
| Water | QS | QS | QS |
| Salicylic Acid | 2.00% | 1.50% | 1.00% |
| Decyl Glucoside | 12.00% | 9.00% | 6.00% |
| Polyquaternium-67 (SoftCAT SL-5) | 1.50% | 1.50% | 1.50% |
| Citric Acid | 0.60% | 0.60% | 0.60% |
| Fragrance | — | 0.50% | 0.50% |

All percentages disclosed herein, unless otherwise stated, are by weight of the named material itself that is found in the compositions, thereby excluding for example the weight associated with carriers, impurities and by-products found in the raw material.

It should be understood that every maximum numerical limitation given throughout this specification includes every lower numerical limitation, as if such lower numerical limitations were expressly written herein. Every minimum numerical limitation given throughout this specification includes every higher numerical limitation, as if such higher numerical limitations were expressly written herein. Every numerical range given throughout this specification includes every narrower numerical range that falls within such broader numerical range, as if such narrower numerical ranges were all expressly written herein.

All parts, ratios, and percentages herein, in the Specification, Examples, and Claims, are by weight and all numerical limits are used with the normal degree of accuracy afforded by the art, unless otherwise specified. The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm” All measurements are performed at 23° C., unless otherwise specified.

All documents cited in the DETAILED DESCRIPTION OF THE INVENTION are, in the relevant part, incorporated herein by reference; the citation of any document is not to be construed as an admission that it is prior art with respect to the present invention. To the extent that any meaning or definition of a term or in this written document conflicts with any meaning or definition in a document incorporated by reference, the meaning or definition assigned to the term in this written document shall govern.

Except as otherwise noted, the articles “a,” “an,” and “the” mean “one or more.”

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition comprising:
a. from about 0.1% to about 3% of a cationic polysaccharide, wherein said cationic polysaccharide is hydrophobically modified with a hydrophobic substituent and a cationic substituent;
b. from about 5% to about 30% of a lathering surfactant;
c. from about 50% to about 90% of a carrier comprising water; and
d. optional adjunct ingredients.

2. The personal care composition of claim 1, wherein the level of said hydrophobically modified cationic polysaccharide is from about 0.25% to about 2.5%.

3. The personal care composition of claim 1, wherein the hydrophobically modified cationic polysaccharide comprises a cellulose ether which has from 4,000 to 10,000 anhydroglucose repeat units and which is substituted with
(a) on the average from 0.0003 to 0.08 moles, per mole of anhydroglucose unit, of a substituent comprising an alkyl or arylalkyl group having from 8 to 24 carbon atoms and
(b) a substituent having the formula [R⁵R⁶R⁷R⁸N⁺](A^{z-})_{1z}
wherein R⁵, R⁶ and R⁷ each independently are —CH₃ or —C₂H₅, R⁸ is —CH₂—CHOH—CH₂— or —CH₂CH₂—, A^{z-} is an anion, and z is 1, 2 or 3.

4. The personal care composition of claim 3, wherein said hydrophobically modified cationic polysaccharide comprises a cellulose ether selected from the group consisting of: hydroxyethyl cellulose, hydroxypropyl cellulose, methyl cellulose, hydroxypropyl methyl cellulose, hydroxyethyl methyl cellulose, hydroxyethyl carboxylmethyl cellulose, or mixtures thereof.

5. The personal care composition of claim 4, wherein said cellulose ether comprises a hydroxyethyl cellulose.

6. The personal care composition of claim 5, wherein said cellulose ether comprises on the average from 1.0 to 3.0 moles of hydroxyethyl groups per mole of anhydroglucose unit.

7. The personal care composition of claim 5, wherein said cellulose ether comprises on the average from 0.0005 to 0.07 moles of the substituent (a), per mole of anhydroglucose unit.

8. The personal care composition of claim 5, wherein said cellulose ether comprises on the average from 0.1 to 0.6 moles of the substituent (b), per mole of anhydroglucose unit.

9. The personal care composition of claim 5, wherein the substituent (a) has the formula:
R¹R²R³R⁴N⁺(A^{z-})_{1/z}
wherein R¹ and R² each independently are —CH₃ or —C₂H₅, R³ is —CH₂—CHOH—CH₂— or —CH₂CH₂—, R⁴ is an alkyl or arylalkyl group having from 8 to 24 carbon atoms, and A^{z-} is an anion, and z is 1, 2 or 3.

10. The personal care composition of claim 1, further comprising a lubricant selected from: a lubricious water soluble polymer; a water insoluble particle, a hydrogel forming polymer, and a mixture thereof.

11. The personal care composition of claim 10, wherein said lubricious water soluble polymers comprises at least one of a polyethylene oxide, a polyvinylpyrrolidone, a polyacrylamide, and a mixture thereof.

12. The personal care composition of claim 10, wherein said water insoluble particles comprises at least one of an inorganic particles, an organic polymer particles, and a mixture thereof.

13. The personal care composition of claim 10, wherein said hydrogel-forming polymers comprises at least one of: a polyacrylic acid or polymethacrylic acid partially esterified with a polyhydric alcohol; a hydrophilic polyurethanes; a lightly crosslinked polyethylene oxide; a lightly crosslinked polyvinyl alcohol; a lightly crosslinked polyacrylamide; a hydrophobically modified hydroxyalkyl cellulose; a hydroxyethyl methacrylate; and crosslinked hyaluronic acid.

14. The personal care composition of claim 1, further comprising from about 1% to about 20% of at least one polar solvent.

15. The personal care composition of claim 14, wherein said polar solvent is selected from the group consisting glycerin, 1,3-butylene glycol, propylene glycol, hexylene glycol, propane diol, ethylene glycol, diethylene glycol, dipropylene glycol, diglycerin, sorbitol, and a mixture thereof.

16. The personal care composition of claim 1, wherein said lather surfactant comprises from about 5% to about 20% of alkyl sulfonates, pareth sulfonates, sulfobetaines, alkylhydroxysultaines, alkyl glucosides, alkyl polyglucosides and mixtures thereof.

17. The personal care composition of claim 1, further comprising from about 0.5 to about 2.0% of a salicylic acid.

18. The personal care composition of claim 17, having a compositional pH of from about 2.5 to about 4.5.
